(19) United States
(12) Patent Application Publication
(10) Pub. No.: US 2005/0059576 A1
(43) Pub. Date: Mar. 17, 2005

(54) Targeted delivery of antiviral compounds through hemoglobin bioconjugates

(76) Inventors: J. Gordon Adamson, Georgetown (CA); David Bell, Oakville (CA); Nancy Ng, Vaughan (CA); Pieter Biessels, Toronto (CA)

Correspondence Address: BERESKIN AND PARR, SCOTIA PLAZA, 40 KING STREET WEST-SUITE 4000 BOX 401, TORONTO, ON, M5H 3Y2, CA

(21) Appl. No.: 10/846,597

(22) Filed: May 17, 2004

Related U.S. Application Data
(63) Continuation-in-part of application No. 10/231,062, filed on Aug. 30, 2002.
Application No. 10/231,062 is a continuation of application No. 09/302,351, filed on Apr. 30, 1999, now Pat. No. 6,479,637.
(60) Provisional application No. 60/470,445, filed on May 15, 2003.
(60) Provisional application No. 60/513,575, filed on Oct. 24, 2003.

(30) Foreign Application Priority Data
Apr. 30, 1998 (CA) 2,236,344

(51) Int. Cl. A61K 38/42; A61K 31/7056

(52) U.S. Cl. 514/6; 514/43

(57) ABSTRACT

This invention relates to targeted delivery of anti-viral compounds through protein bioconjugation. More particularly, it relates to an anti-viral compound conjugated to a protein, such as hemoglobin and to a method of treating a viral infection using said conjugate. The invention also provides a method of targeted drug delivery of an anti-viral nucleoside analogue to macrophages, cells comprising a hemoglobin receptor and to CD163 bearing cells.

### RELATED APPLICATIONS

This application is a continuation-in-part of U.S. patent application Ser. No. 10/231,062, filed Aug. 30, 2002, entitled “Hemoglobin-Haptoglobin Complexes”, which is a continuation of U.S. patent application Ser. No. 09/302,351 filed Apr. 30, 1999, now U.S. Pat. No. 6,479,637, issued Nov. 12, 2002, which in turn claimed priority from Canadian patent application number 2,236,344, filed Apr. 30, 1998. This application further claims the benefit of priority from U.S. provisional patent application No. 60/470,445, filed May 15, 2003, entitled “Hemoglobin-Ribavirin Conjugate For The Treatment of Viral Infections” and 60/513,575, filed Oct. 24, 2003, entitled “Ribavirin Conjugates and Targeted Drug Delivery”. All of these applications are incorporated herein by reference. As the one year anniversary from the May 15, 2003 priority date is May 15, 2004 and falls on a Saturday, this application is being filed on the next available business day, which is Monday, May 17, 2004.

### FIELD OF THE INVENTION

This invention relates to the field of targeted drug delivery. In another aspect, the invention relates to a method of targeted drug delivery of anti-viral compounds. In another aspect the invention relates to targeted drug delivery of hemoglobin-anti-viral compound conjugates, to said conjugates per se, and to methods and uses thereof, including but not necessarily limited to a method of treatment of viral infections and related conditions using said conjugates.

### BACKGROUND OF THE INVENTION

Targeted Drug Delivery

The importance of targeted drug delivery to enhance treatment regimes is well known in the art. Drug formulations (e.g. drug carriers used, pill, vs. liquid form, pill coatings) and mode of delivery (e.g. intravenous, oral, inhalation) can have an impact on the effectiveness of a drug and its side effects. However, optimization of a treatment regime using targeted drug delivery is a challenge. A number of factors need to be taken into account; knowledge of the condition to be treated, mechanism of action of a drug, identification of a target site for the drug and a suitable carrier or formulation to enable the delivery of drugs to the target site in a manner wherein the drug retains its activity.

One targeted drug delivery technique developed in recent years is receptor- mediated delivery. This has the advantage of high specificity of delivery to the cells which express a receptor for the drug carrier (ligand).

The specific targeting of low molecular weight therapeutic and diagnostic agents to tissues is enhanced greatly through the use of receptor-mediated delivery. Diagnostic agents such as fluorescent or radiolabeled substances can be used to indicate the location and quantity of cells bearing the targeted receptors when such agents are administered as complexes with ligands for those receptors. These complexes are also useful in characterizing the binding and transport properties of receptors on cells in culture. Such information is useful in detection of, and/or design of therapy for, tissues containing the target cells, either in vitro or in vivo. However, it is still a challenge to identify optimal targets and effective modes of delivery to desired targets.

Certain targeted drug delivery means have been identified- For instance, means of drug delivery to macrophages that have previously been described, include, e.g., liposomes, gold, gold-labeled liposomes, polystyrene or carbon particles, macrophagespecific antibodies, microspheres, nanoparticles, lipoproteins, erythrocytes, and pathogens known to infect macrophages. (See for example, U.S. Pat. Nos. 6,599,887; 6,448,932; 6,096,311; 6,071,517; 6,018,031 and 4,764359; and Schmidt, J. et al., Brain (2003) 126(8): 1895-904). In another embodiment, carriers modified by polyethylene glycol (PEG), amphiphiles, peptides or proteins such as fibronectin, tuftsin, gelatin, or glycosylated carriers may also be used. However, all of said delivery methods have limitations. For instance, there can be leakage of drugs with use of liposomes or microspheres, further, solubility, heterogeneity of size, biodistribution, and toxicity, biocompatibility with artificial components and cost.

Hemoglobin Hemoglobin, as a natural component of red blood cells, is present and circulating throughout the body in relatively large quantities. Hemoglobin has well-established bloacceptability and clearance mechanisms, and the potential to transport drugs through the circulatory system.

Thus, Kluger et al., U.S. Pat. No. 5,399,671 describes a hemoglobin compound which has been cross-linked to effect intramolecular stabilization of the tetrameric structure thereof, but which contains a residual functional group on the cross-linker residue to which drugs for delivery can be covalently attached.

Anderson et al., U.S. Pat. No. 5,679,777, describes complexes of hemoglobin compounds and polypeptide drugs, in which the polypeptide drug is bound to a globin chain through a disulfide linkage to a cysteine unit inherent in, or genetically engineered into, the globin chain.

Haptoglobins (Hp) constitute part of the α₂-globin family of serum glycoproteins. Haptoglobins are present in mammalian plasma, and constitute about one-quarter of the α₂-globulin fraction of human plasma. Each individual has one of three phenotypic forms of haptoglobin, of close structural and chemical identity. Haptoglobins are composed of multiple αβ dimers and the phenotypes are conventionally denoted Hp 1-1, Hp 2-1 and Hp 2-2 The β chains are identical in all haptoglobin phenotypes, but the α chains vary (α¹ and α²). The amino acid sequences of all chains are known. Hp 1-1 is composed of two α¹β dimers and has a molecular weight of about 98 kDa. The structure of Hp 2-1 and Hp 2-2 can be written as follows: (α¹β)₂ (α²β)ₙ where n=0,1,2, . . . and (α²μ)ₘ where m=3,4,5, . . . respectively.

One function of haptoglobin is to bind extracellular hemoglobin, arising from red blood cell lysis, to form essentially irreversible haptoglobin-hemoglobin complexes that are recognized by specific receptors Hemoglobin-haptoglobin receptors have been identified on hepatocytes in the liver and more recently on macrophages. In this way, hemoglobin is targeted to the liver or macrophages for metabolism. Further, CC163 has also been identified as a hemoglobin-haptoglobin receptor on macrophages (Kristiansen et al, Identification of the haemoglobin scavenger receptor. Nature 409, 198-201 (2001)). In one aspect, hemoglobin-haptoglobin can be targeted to cells containing CD163 on their cell surface, such as macrophages.

Anti-viral Therapy

Currently, there are limited options in the treatment of viral conditions. Because viruses incorporate into the infrastructure of the host cell, developing drugs that are specific or have a sufficient specificity to viral infected cells with minimum toxicity to non-infected host cells is a challenge. A therapeutic index which is minimum toxicity dose to a host cell divided by the minimum effective dose that is toxic to a virus that favours the use of the anti-viral is desirable. One example of a suitable range for the therapeutic index is 100-1000, but this invention is not bound by such a range.

Many viruses encode for their own RNA/DNA polymerases or other proteins or enzymes necessary for their replication or function, such as proteases, mRNA capping enzymes, neuramidases, ribonucleases, and kinases. Samples of such viruses include, but are not necessarily limited to Hep B, Poz, Irido, herpes, Adeno, Corona, Rhabdo, Paramyxo, Orthomyxo, Toja, Reo nand Picoma viruses. Anti-viral therapy often targets nucleic acid synthesis. This can be affected in a number of ways, for instance, where viral polymerases are more sensitive to the drug than the host enzymes. Thymidine kinase is one enzyme that is encoded by some viruses, and can activate drug to toxic form, wherein uninfected cells do not. So some anti-virals can be administered in pro-drug form and designed to be activated in cells comprising said viral thymidine kinase. Administering drugs in a non-phosphorylated form, can make it easier for the drug to enter a cell, whose membrane is poorly permeable to phosphorylated drugs The drug can then be phosphoylated to an active form by the thymidine kinase. Alternatively, one could administer the drug in an active phosphorylated form, where mode of delivery permits, in which case the drug would be active without further processing.

One class of anti-viral agents that has been used is nucleoside analogues. Nucleoside analogues have an altered sugar, base or both. Examples of nucleoside analogues includeidoxuridine, acyclovir (acycloguansoine), ganiciclovir, adensosine arabinoside (AraA, Vidarabine), Ara-AMP, AraC (cytarabine), Ara-CMP, azidothymidine (AZT), ribavirin, didenosine (DDI), dideoxycytosine (DDC), stavudine (d4T), Epivir (3TC), abacavir (ABC), iodo-deozyuridine (DU), Valacyclovir, and bromovinyl deoxiuridine (BVDU).

Nucleoside analogs that include sugar modifications are acyclovir (a guanosine analogue), ganiciclovir (a 2′-deoxyguanosine analogue, similar to acyclovir but with an extra hydroymethyl group on its side chain), Valacyclovir is the hydrochloride salt of l-valyl ester of acyclovir, AraA, DDI, DDC. Nucleoside analogues with base modifications include DU, BVDU, and Ribavirin which is a guanosine analogue.

Most of the anti-viral nucleoside analogues target nucleic acid synthesis, or thymidine kinase. Resistance or viral mutations to overcome the therapies can develop. Further, delivery of an effective amount in a suitable time period to a desired site while minimizing side effects has been a challenge in anti-viral therapy. Administered alone, the uptake of these class of anti-virals tends to at least some degree be non-specific and are associated with a number of toxic side effects including hemolytic anemia.

Nucleoside analogues of guanosine have been developed and include acyclovir, ganiciclovir, Valacyclovir and ribavirin One such guanosine analogue is ribvairin. Cyclic guanosine analogs have been found to be useful substrates for varicella-zoster (A R Karlstrom, et al., Antimicrob Agents Chemother. 1986 January, 29(1):171-174). They have also been shown to be effective against cytomegalavirus (CMV) (B. Watinen, A. Larsson, U. Ruden, A. Sundquist, E. Solver, 1987 February, 31(2):317-320).

Ribavirin

Ribavirin is a known nucleoside analogue and is used in anti-viral treatment against a wide range of RNA and DNA viruses. It is an analogue of guanosine (Hoffman et al, 1973, Antimicrob. Agents Chemother. 3:235; Sidwell et al, 1972, Science 177:205) that was developed by ICN Pharmaceuticals Inc. It is approved for the treatment of infections caused by RSV (respiratory syncitial virus) and HCV (hepatitis C virus). Current combination therapy to treat HCV involves use of interferon-alpha (IFNα) and ribavirin Ribavarin is also one of the only drugs that was used in the treatment for SARS (severe acute respiratory syndrome). Ribavirin by itself is ineffective as an anti-viral agent in the treatment of HCV infection, but combined with IFNα, increases the rate of sustained viral response. However, it takes 4 weeks of dosing to achieve steady state plasma levels of ribavirin and ribavirin is taken up non-specifically by all body tissues. As such, current treatments require daily administration of high doses (800-1200 mg/day) for periods of 24-48 weeks. This is not practical for treatment of non-chronic conditions, such as acute disease like SARS and the like. Further, current treatment of HCV infections using ribavirin is limited by ribavirin toxicity. The most frequent side effect of ribavirin is the development of hemolytic anemia, which occurs in ˜10% of patients. Generally, hemoglobin levels decrease by 3 g/dL or more in 54% of all patients. The percentage of patients who achieve a sustained viral response using pegylated IFNα and ribavirin is at best 50-60%. The ribavirin toxicity can result in either dose reduction or its discontinuation in certain patients, with a consequent reduction in response to therapy. The mechanism for the beneficial action of ribavirin is not entirely understood, as ribavirin appears not to eradicate viral replication.

As such there is a need for improved anti-viral nucleoside analogue treatment and therapy. There is also a need for improved delivery and targeted delivery of said anti-viral nucleoside analogues, such as ribavirin, AraA and AraC and similar anti- viral drugs and for an improved toxicity profile.

### SUMMARY OF THE INVENTION

In one aspect, the present invention provides an improved method of treatment of viral conditions comprising the use of hemoglobin and an anti-viral drug conjugate; to use of a hemoglobin-anti-viral nucleoside analogues and to hemoglobin-anti-viral nucleoside conjugates per se. Haptoglobin complexes with said conjugates and pharmaceutical compositions comprising said conjugates and/or complexes are also provided. Methods of diagnosis and drug screening and of determining optimal therapeutic regimes are also provided.

The invention relates to the field of targeted drug delivery and to an enhanced method of delivering drugs, such as nucleoside analogues, analogues thereof, prodrugs thereof or pharamceutically acceptable salts thereof to cells capable of binding hemoglobin. In another aspect, the invention provides an enhanced method of anti-viral treatment, by targeting anti-viral drug delivery to cells expressing receptors for hemoglobin or its derivatives, or hemoglobin-haptoglobin, such as CD163, or to macrophages. In another aspect the invention provides an enhanced method of ribavirin or ribavirin-like anti-viral therapy by targeting its delivery to CD163 or other receptors interacting with Hb. comprising cells and macrophages. In one aspect, the invention provides a pharmaceutical composition comprising a drug (e.g. anti-bacterial, anti-viral, or other substance (e.g. a diagnositic substance), but preferably a nucleoside analogue, for instance, ribavirin and a pharmaceutically acceptable carrier, wherein said carrier directs delivery of ribavirin or said ribavirin analogue to CD163 containing cells and to macrophages. In a preferred embodiment, the pharmaceutically acceptable carrier is hemoglobin, preferably a mammalian hemoglobin, more preferably bovine or human hemoglobin, most preferably human hemoglobin. In another embodiment, the hemoglobin can be isolated and purified from red blood cells or from cell culture. In yet another embodiment, the hemoglobin can be recombinant hemoglobin. In another embodiment, the hemoglobin may be of natural sequence or a variant thereof, including truncated or composite sequences.

In another preferred embodiment, the hemoglobin is conjugated to the drug, anti-viral nucleoside analogues, such as AraA, AraC or preferably ribavirin, directly or indirectly through a linker. As such, in one embodiment the invention provides a hemoglobin-ribavirin conjugate. In yet another embodiment, the hemoglobin is non-intramolecularly cross-linked. In one embodiment, the hemoglobin is non-intra and non-inter-molecularly crosslinked. In another embodiment, the hemoglobin drug conjugate is capable of binding haptoglobin. In another embodiment, the drug is conjugated to the hemoglobin at a site independent of the haptoglobin binding site- The drug may also be conjugated to the haptoglobin portion of the hemoglobin-haptoglobin complex.

The invention also provides hemoglobin—drug conjugates that are also bound or that are capable of binging to haptoglobin. In one embodiment, the haptoglobin-hemoglobin-drug complex is capable of uptake by cells expressing hemoglobin-haptoglobin receptors, such as CD163. As such, in one embodiment the hemoglobin-ribavirin conjugates and hemoglobin-ribivirin analogue conjugates can be targeted to macrophages and used in the treatment of viral infections, such as coronavirus, RSV, HCV or the like. It can also be used to target hepatocytes and used in the treatment of conditions in such cells such as viral hepatitis.

In another aspect the invention provides a method of treating a viral infection comprising administering to a patient in need thereof, a pharmaceutical composition comprising an anti-viral substance, such as ribavirin or a ribavirin analogue and a pharmaceutically acceptable carrier wherein said carrier directs delivery of said anti-viral to macrophages.

In one embodiment, the viral infection is selected from the group consisting of MHV-3, Hepatitis C, HIV, SARS, RSV, coronavirus.

In another embodiment, the invention provides a method of treating a condition that is modulated through macrophages (i.e. modulation of inflammatory and immune responses), comprising administering to a patient in need thereof, a pharmaceutical composition comprising a drug, for example, ribavirin or ribavirin analogue, and a pharmaceutically acceptable carrier, wherein said carrier directs delivery of the drug to macrophages. In one embodiment the pharmaceutically acceptable carrier is hemoglobin. In another embodiment, the hemoglobin is conjugated to the drug, for example ribavirin. In a further embodiment, the drug is an agent capable of modulating macrophage function, for example, the agent is capable of modulating the immune response or the agent is capable of modulating the inflammatory response. In more specific embodiments, macrophage function is modulated through the secretion of specific cytokines such as IFN, such as IFNγ, or TNFα, or through the expression of certain surface molecules such as CD163, MHC class II and associated co-stimulatory and adhesion molecules. As such, in another embodiment, the invention provides a method of treating macrophage-mediated conditions such as non-viral infectious agents/pathogens/parasites e.g., tuberculosis, bacillus anthraxis and its spores.

In another embodiment, the invention provides a method for targeted delivery of drugs, such as ribavirin to CD163 or other hemoglobin-haptoglobin receptor bearing cells, such as macrophages or hepatocytes. In one embodiment, the invention provides a method for targeted delivery of drugs, such as ribavirin to macrophages. Further the invention provides a method to modify macrophage related immune response and/or inflammatory response. Further the invention provides a method of modulating macrophage response through modification of cytokine secretion, and/or protein expression. The invention also provides a method for delivering components of hemoglobin-haptoglobin as drugs (protein, heme, iron) to modulate macrophages or receptor-bearing cells. All these methods can be affected by administering an effective amount of the compositions and/or conjugates and/or complexes of the present invention to a subject in need thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description

### BRIEF DESCRIPTION OF THE DRAWINGS

The invention will now be described in relation to the drawings in which:

FIG. 1 illustrates the method used to prepare hemoglobin ribavirin conjugates of the invention in one embodiment of the invention.

FIG. 2 Anion-exchange HPLC of Hb-ribavirin conjugate using a pH gradient (pH 8.5-6.5) under non-denaturing conditions.

FIG. 3 is a MALDI-TOF mass spectrometry profile of a Hb-ribavirin conjugate. The increase in MW of each of the α and β globin chains by multiples of the ribavirin phosphate mass (308 Da) confirms addition of multiple drugs per globin chain.

FIG. 4 illustrates biological activity of ribavirin enzymatically cleaved from Hb-ribavirin conjugate. Human HepG2 (left-A) and mouse AM12 (right-B) hepatic cells were treated with ribavirin (□), and ribavirin cleaved and purified from Hb-ribavirin conjugate (▪). The cells were assayed for uptake of bromodeoxyuridine (BrdU) as a measure of cell proliferation. Inhibition of cell proliferation is represented as % inhibition of control untreated cells.

FIG. 5 illustrates in vitro uptake of Hb-ribavirin conjugate by hepatic and non-hepatic cells. Human hepatoma HepG2, mouse AM12 hepatocyte, and 5637 bladder carcinoma cells were assayed for internalization of fluorescein-labeled Hb and Hb-ribavirin conjugate at 37° C. for 2 hr. Results are expressed as fold increase in relative fluorescence units (RFU) over equivalent samples not treated at 37° C.

FIG. 6 is a bar graph illustrating the clinical behaviour of MHV-3 infected mice by plotting the composite clinical score versus days post infection for PBS, hemoglobin-ribavirin conjugate and ribavirin alone treated mice. Scores range from zero (dead) and 1 (poor) to 5 (normal).

FIG. 7 is a micrograph illustrating the histopathogy results for day 3 livers in PBS control, hemoglobin-ribavirin and ribavirin treated, MHV-3 infected mice. It illustrates the degree of liver necrosis and fibrin deposition after infection with MHV-3 in the three treatment regimes.

FIG. 8 is a bar graph illustrating the survival of MHV-3 coronavirus-infected mice under the three treatement regimes: (1) Control (PBS); (2) haptoglobin-hemoglobin-ribavirin (HRC203); and (3) ribavirin

FIG. 9 is a bar graph illustrating the mean viral titer in livers of MHV-3 infected mice (PFU/gm versus days post-infection) for the three treatment regimes: (1) Control (PBS); (2) haptoglobin-hemoglobin-ribavirin (HRC203); and (3) ribavirin.

FIG. 10 is a bar graph illustrating the viral titer in macrophages infected in vitro with MHV-3 under the three treatment regimes: (1) Control (PBS); (2) haptoglobin-hemoglobin-ribavirin (HRC203); and (3) ribavirin.

FIG. 11 is a bar graph illustrating the percent inhibition of viral replication in macrophages infected in vitro with MHV-3 under the three treatment regimes: (1) Control (PBS); (2) haptoglobin-hemoglobin-ribavirin (HRC203); and (3) ribavirin.

FIG. 12 is a bar graph illustrating that hemoglobin-ribavirin conjugate decreases cytokine production in vitro in macrophages infected with MHV-3: (A) TNFα and (B) IFNγ.

FIG. 13 is a graph of the HPLC results illustrating the conversion of Ara-C to the imidazolide derivative.

FIG. 14 is a graph of the anion exchange chromatography results illustrating the formation of Hb-Ara-A (A) and Hb-Ara-C (B) conjugates

FIG. 15 is a graph of the size exclusion chromatography results illustrating Hp binding of Hb-Ara-A (A) and Hb-Ara-C (B),. ˜32 kDa Hb species (Hb-drug conjugates) elute at approximately 38 minutes. Polymers of these elute as an earlier shoulder to this peak. Hp complexes elute in the 20-25 minute range.

FIG. 16 is a graph illustrating the uptake of fluorescently labeled Hp, Hp-Hb and Hp-Hb-AraA on wild type (WT) CHO and CD163 Expressing (CD163) CHO cells.

### DETAILED DESCRIPTION

In one aspect, the present invention provides a pharmaceutical composition comprising an anti-viral nucleoside analog and a pharmaceutically acceptable carrier comprising hemoglobin.

In another aspect, the present invention provides a use of an anti-viral nucleoside analogue in the preparation of a medicament comprising a hemoglobin as a pharmaceutically acceptable carrier. In aspect said medicament comprising a hemoglobin- anti-viral nucleoside analogue conjugate. In another aspect, said medicament can be used in the treatment of a viral condition. In another aspect, said medicament can be used to target delivery of the medicament to a cell comprising a hemoglobin or hemoglobin-haptoglobin receptor, such as a CD163 bearing cell or macrophage. In another aspect, said conjugate in the medicament is capable of binding to or is bound to haptoglobin.

In another aspect, the present invention provides a method of enhancing the use of an anti-viral nucleoside analogue in the treatment of a viral infection by conjugation of the said anti-viral nucleoside analogue to hemoglobin and administering said hemoglobin-anti-viral nucleoside analogue conjugate to a subject in need thereof. The present inventors have shown that not only can the delivery of such hemoglobin-anti-viral conjugates be targeted to cells comprising a hemoglobin or a hemoglobin-haptoglobin receptor, they can also improve the efficacy and safety of said anti-viral nucleoside analogue over the use of said anti-viral nucleoside analogue alone. The present invention enables the use of a reduced dose of a nuclcoside analogue as compared to a control, such as the use of the nucleoside analogue alone, in the treatment of a viral condition.

In one aspect, the present inventors have also shown that such hemoglobin-anti-viral conjugates can be directed to cells comprising a hemoglobin receptor, such as a hemoglobin-haptoglobin receptor. In one aspect, the inventors have shown that said hemoglobin-anti-viral nucleoside analogue conjugate can be targeted for delivery to CD163 bearing cells and to macrophages and can enhance the delivery of said nucleoside analogue to said cells.

In another aspect, the present invention provides a method of treating or reducing anemia, such as hemolytic anemia, that is often associated with nucleoside analogue therapy, using the hemoglobin-anti-viral nucleoside analogue conjugate of the present invention, by administration of said conjugate, such as an effective amount of said conjugate, to a subject in need thereof.

In another aspect the hemoglobin-anti-viral nucleoside analogue conjugates of the present invention can be used as immunomodulators, for instance to decrease the macrophage mediated immune response, such as that associated with TNF-α or IFN, such as IFN-γ, expression.

In one aspect, although the nucleoside analogues used in the invention may have anti-viral properties, the conjugates of the invention can be used for any use found for said nucleoside analogue, or hemoglobin-nucleoside analogue conjugate, for instance in the treatment of other medical conditions. Such other medical conditions may include hemoglobin-haptoglobin receptor-comprising cell-mediated, CD163 bearing cell-mediated or macrophage-mediated medical condition.

It should be noted that the use of said hemoglobin-anti-viral nucleoside analogue conjugates is not intended to be limited by any particular mechanism of action or pathway.

In one aspect, the nucleoside analogue is selected from the group consisting of AraA, AraC and guanosine analogues, such as ribavirin or analogues and derivatives thereto, obvious chemical equivalents thereof and functional equivalents thereof. The nucleoside analogue can also be a pharmaceutically acceptable salt of said analogues.

In one aspect the conjugate is capable of binding to haptoglobin or is further bound to haptoglobin to form a haptoglobin-hemolgobin-anti-viral nucleoside analogue complex. Said haptoglobin can be directly or indirectly (e.g., through a linker) to said hemoglobin In one aspect said complex is formed in a way that permits binding of the complex to a hemoglobin-haptoglobin receptor. In another aspect, the invention is directed to a method of treating a hemoglobin-haptoglobin receptor-comprising cell-mediated or macrophage-mediated medical condition, comprising the use of a hemoglobin-anti-viral nucleoside conjugate. In another aspect, the invention provides a pharmaceutical composition comprising a hemoglobin-anti-viral nucleosides analogue conjugate.

In one embodiment, the hemoglobin is conjugated to the anti-viral nucleoside analogue, either directly or through a linker. The hemoglobin can be non-intramolecularly cross-linked, or cross-linked hemoglobin (intra-and/or inter-molecularly cross-linked). In another embodiment, the hemoglobin is human hemoglobin.

In one preferred embodiment of the invention, the anti-viral nucleoside of the conjugate is a ribavirin. The conjugate and composition of the present invention can be used to treat any condition that the ribavirin can be used for whether it is for the treatment of a viral or a non-viral infection or other associated conditions such as hemolytic anemia, or immunoregulatory conditions, such as autoimmune disorders where immunosuppression or suppression of TNF-α or IFN, such as IFN-γ, is desired.

It should be noted that the hemoglobin-anti-viral nucleoside analogue conjugates of the present invention can be used alone or in the preparation of a medicament for the treatment of medical conditions as noted herein, and/or in combination therapies. For instance, the hemoglobin-ribavirin conjugates of the present invention can be used in combination with IFN, such as IFN-γ, therapy.

Hemoglobin

The hemoglobin compound useful as a component of the conjugates of the present invention can be substantially any hemoglobin compound providing the necessary degree of biocompatibility for administration to a patient or animal, the necessary sites for attachment of the drug or other substance of interest, and preferably having sufficient binding affinity for haptoglobin. Within these limitations, it can be a naturally occurring hemoglobin from human or animal sources. It can be a non-intramolecularly cross-linked hemoglobin. It can be a modified natural hemoglobin, e.g. an intramolecularly cross-linked form of hemoglobin to minimize its dissociation into dimers, an oligomerized (intra- and/or non-intra-molecularly cross-linked oligomers) form or a polymerized form. It can be a hemoglobin derived from recombinant sources and techniques, with its naturally occurring globin chains or such chains mutated in minor ways It can be comprised of subunits or fragments of Hb, or derivatives thereof, which have affinity for haptoglobin. It can be a hemoglobin in which individual amino acids of the globin chains have been removed or replaced by site specific mutagenesis or other means. In one embodiment of the invention, certain modifications which are known to decrease the affinity of hemoglobin for binding to haptoglobin are in one embodiment of the invention, preferably avoided in hemoglobin compounds used in the present invention. Modifications to hemoglobin and/or haptoglobin that enhance haptoglobin binding to hemoglobin or the conjugate of the invention or binding of the conjugate or complexes of the invention to a cell or particular cell type are encompassed within the scope of the present invention.

One type of hemoglobin compounds are those which comprise hemoglobin tetramers intramolecularly cross-linked to prevent their dissociation into dimers, and which leave functional groups available for chemical reaction with the drug or other substance, either directly or through a chemical linker molecule. Such hemoglobin compounds provide a known, controlled number of reactive sites specific for the therapeutic substance of interest, so that an accurately controlled quantity of the therapeutic substance can be attached to a given amount of hemoglobin compound. They also avoid utilizing sites on the globin chains for linkage to the therapeutically active substance drug or other substance, so as to minimize conformation disruption of the globin chains and minimize interference with the hemoglobin-haptoglobin binding and with binding of the construct complex to the receptor.

Human hemoglobin, e.g. that is obtained from outdated red blood cells, and purified to a desired level, such as by the displacement chromatography process described in U.S Pat. No. 5,439,591 Pliura et al. is one raw material that can be used for preparation of the hemoglobin product for use in the conjugate and/or complex of the present invention. In one embodiment, this material may be cross-linked with a trifunctional cross-linking agent as described in aforementioned U.S. Pat. No. 5,399,671, Kluger et al., namely a reagent which utilizes two of its functional groups for intramolecular cross-linking between subunits of the hemoglobin tetramer, and leaves its third functional group available for subsequent reaction with a nucleophile. A specific example of such a cross-linking reagent is trimesoyl tris(3,5-dibromosalicylate), TTDS, the preparation of which is described in the aforementioned Kluger et al. U.S. Pat No. 5,399,671.

The anti-viral nucleoside analogue can be bound to the hemoglobin, either directly or through a chemical linker or spacer, and then this complex may be administered to the patient so that the haptoglobin-hemoglobin binding takes place in vivo The entire haptoglobin-hemoglobin-drug complex can, if desired, be formed extracorporeally and then administered to the patient, and this can under some circumstances lead to better control of the amounts of active substance finally being delivered to the hemoglobin or hemoglobin-haptoglobin receptor bearing cells, such as CD163 bearing cells, or macrophages. However, such a procedure is not normally necessary, save for those exceptional patients having zero or low levels of haptoglobin, e.g. in conditions of acute hemolysis. Such patients can be administered haptoglobin before, during and/or after administration of the hemoglobin-drug conjugate of the invention. Usually, however, there is sufficient haptoglobin in the patient's plasma to form the haptoglobin-hemoglobin-drug complex in situ to effect its delivery to the target cells. Preparation of the hemoglobin-anti-viral nucleoside analogue and administration of that to the patient, to form the haptoglobin-hemoglobin-anti-viral nucleoside complex in situ is generally cheaper and less complicated.

Use of intramolecularly crosslinked hemoglobins will give rise to high molecular weight polymers containing more than one hemoglobin and/or haptoglobin owing to the presence of multiple binding sites on each of these proteins. There may be advantages to using noncrosslinked hemoglobin as a component of the conjugates and -complexes of the present invention. Such a hemoglobin, with a drug or other substance bound to it, will dissociate into dimeric hemoglobin of approximate molecular weight 32 kDa, and two such dissociated dimeric hemoglobin products bind to a single molecule of haptoglobin to give a complex according to the present invention. The formation of high molecular weight haptoglobin-hemoglobin complexes is thus avoided. Haptoglobin binding to αβ-dimers is generally a much faster reaction than haptoglobin binding to crosslinked hemoglobin. The lower molecular weight complexes resulting from the use of non-crosslinked hemoglobin may show improved receptor binding and uptake.

Where hemoglobin of a form which will dissociate into dimers is used as a component of the present invention, or where hemoglobin dimers themselves are used, for example, where the dimers have been modified such that they cannot reform 64 kDa hemoglobin, the haptoglobin-hemoglobin-antiviral nucleoside analogue conjugate-complex can be formed according to the invention extracorporeally, and then the finished conjugate-complex is administered to the patient, so as to avoid the risks attendant on administering to the body a molecular species of too small a molecular weight, namely, clearing the drug too rapidly through excretion. Administration of Hb dimers bearing anti-virals may be possible without prior binding to haptoglobin in cases where complex formation in vivo is adequate prior to clearance of the modified dimer.

A further example of a hemoglobin compound useful in conjugate-complexes and conjugates of the present invention is dimeric hemoglobin bearing a modifying group containing thiol, preferably a terminal side chain thiol, of the type described in U.S. Provisional Patent Application of Kluger and Li, entitled “Hemoglobin With Chemically Introduced Disulfide Crosslinks and Preparation Thereof”, filed Nov. 3, 1997. Appropriate chemistries can be used for attachment of nucleoside analogues, such as ribavirin or ribavirin analogues to such dimeric hemoglobin, either by direct reaction with the exposed thiol, or by direct reaction with an activated form of the thiol, or by mixed disulfide formation, or through a linker molecule. Conjugate-complexes of this type are made extracorporeally and administered to a patient in this form. The drug or other substance conjugate can also be administered for in vivo Hp binding. The use of dissociable hemoglobin (32 kDa molecular weight) has the advantage over the use of cross-linked hemoglobin tetramers in that they provide an exposed dimer-dimer interface which facilitates haptoglobin binding.

The conjugate-complexes and conjugates of the present invention may also utilize hemoglobin which has been modified in a manner which results in impaired nitric oxide binding. Such modified hemoglobins are known in the art. Reduced NO binding may reduce the tendency of the hemoglobin to effect modifications to a patient's blood pressure upon administration, an effect which has been noted with some hemoglobins, even in small amounts In forming the conjugate-complex or conjugate, it may be necessary to interpose between the reactive site on the hemoglobin chosen and the drug, a chemical linker or a spacer group. This depends upon the nature of the available chemical group on hemoglobin for linking, and on the chemical groups available on the substance to be bound to hemoglobin, for this purpose. For example, ribavirin may be conjugated at one of its ribose hydroxyl groups to an amino group of the hemoglobin through a phosphoramidate linkage Such a linkage may be cleaved enzymatically or otherwise in the target cell and liberate the phosphorylated form of the nucleoside analogue in the cell.

In one embodiment, there may be advantages to using polymerized Hb, such as increased circulation time whether pre-complexed with Hp or not, and in the case of no pre-complexation, then the increased circulation time would possibly allow for more complete complexation in vivo. The larger polyHb-Hp complexes may have altered recognition and uptake by macrophages, also. Both intramolecularly and non-intramolecularly polymerized hemoglobin can be used.

With regard to the formation of hemoglobin-haptogobin drug conjugates, such as the anti-viral nucleoside analogue conjugates of the present invention, such conjugates can be made in accordance with the method disclosed in U.S. Pat. No. 6,479,637, which is incorporated herein in its entirety by reference A conjugate-complex according to one embodiment of the present invention comprises a haptoglobin molecule, which may be haptoglobin 1-1 or any other phenotype, bonded to one or more molecules of a hemoglobin compound by means of strong non-covalent interaction. The hemoglobin may be cross-linked, oligomerized or unmodified, as described above.

For instance, in one embodiment, the hemoglobin-haptoglobin conjugate-complex or conjugate according to the present invention can be generally represented by the formula:
(HP)ₐ-(Hb)_{b}-(Lc-Ad)ₑ
where
a=1 to about 10;
b=0.5 to about 10;
c=0 to about 10;
d=1 to about 20;
e=1 to about 20;

Hp is haptoglobin as described herein;

Hb is a hemoglobin as described herein; in one embodiment, it is a non-intramolecularly cross-linked Hb;

L is a linker as described herein; and

A is a anti-viral nucleoside analogue as described herein, such as ribavirin, araA and araC, or pharmaceutically acceptable salts thereof, in which the stoichiometry of Hp to Hb in the complex is dictated by the available number of binding sites on the two proteins, but is generally of the order of 1:0.5 to 1:2.

In yet another embodiment, the hemoglobin can be oxyhemoglobin, deoxyhemoglobin, carboxyhemoglobin, or met-hemoglobin.

Other forms of hemoglobin or compounds that can deliver drugs to cells comprising hemoglobin-haptoglobin receptors can also be used.

Receptors/Carriers

The hemoglobin-haptoglobin complexes, whether formed in vivo or ex vivo are known to bind to receptors on hepatocytes. Not wishing to be bound by any particular theory or mechanism of action, CD163 receptors, (one form of hemoglobin-haptoglobin receptor) are present on macrophages and can bind hemoglobin-haptoglobin complexes. CD163 is disclosed in Christiansen et al., “Identification of the haemoglobin scavenger receptor” (2001) Nature 409:198-201, published on Jan. 11, 2001 as binding specifically to Hb-Hp, and that this receptor is found on macrophages and certain monocytes. Activation of the CD163 receptor triggers a cascade of intracellular events leading to an anti-inflammatory response. As such the present invention provides hemoglobin conjugated to anti-viral nucleoside analogues that can deliver the anti-viral nucleoside analogues to hepatocytes, macrophages or other cells having hemoglobin-haptoglobin receptors, such as CD163.

Further, the conjugate-complexes or conjugates of the present invention may exert beneficial effects on neighboring cells, if the anti-viral nucleoside analogue that is bound to the hemoglobin is, for example, one which is active towards neighboring cells even if they are not cells having receptors for the hemoglobin-anti-viral nucleoside conjugates or complexes of the present invention. They may also modulate or initiate the activity of other therapeutic or diagnostic agents delivered by other methods for hepatocyte or macrophage modification, such as prodrugs, enzymes or genes coding for enzymes and requiring activation to cause an effect.

In general, when a hemoglobin-anti-viral nucleoside analogue conjugate is used, in one embodiment, several molecules of the anti-viral (e.g., AraA, AraC or ribavirin) are attached to each hemoglobin tetramer, in a manner that still enables haptoglobin binding. If non-intramolecuarly cross-linked, the hemoglobin dissociates into 2 dimers and is tightly bound by the plasma protein haptoglobin. The haptoglobin-hemoglobin-anti-viral nucleoside analogue complex is carried through the bloodstream to cells bearing receptors for the complex and is internalized through an endocytic pathway. Once inside the cell, the nucleoside analogues enzymatically or otherwise cleaved from the complex and released into the cell cytoplasm to exert its biological effect.

In one embodiment of the invention, the hemoglobin-anti-viral nucleoside analogue is a hemoglobin-phophoramidate-anti-viral nucleoside analogue, for instance, such as described in the examples. In this form, the nucleoside analogues may be cleaved from the hemoglobin in phosphorylated form. As such, the nucleoside analogue is released into the cell cytoplasm in an active form or at least in a form that does not require further phosphorylation for its activity

Hemoglobin is used as the drug delivery agent in the examples below, with unexpected enhanced effects.

The conjugates and complexxes of the present inventin can be used in conjunction with other therapies. For instance, hemoglobin-ribavirin conjguates can be used in conjgunction with IFN therapy or treatment.

Anti-viral Nucleoside Analogues

Nucleoside analogues have an altered sugar, base or both. In one aspect, anti-viral nucleoside analogues that can be used in the present invention can be any nucleoside analogue that has anti-viral activity It can include, purine or pyramidine analogues, such as analogues of adenine, guanosine, uracil, thymine or cytosine. In general, analogues are compounds that have structural similarity to the natural occurring nucleosides but differ in certain components and can have similar, enhanced, diminished or opposite effects. The nucleoside analogues of the present invention can also be analogues of the anti-viral nucleoside analogues.

Examples of anti-viral nucleoside analogues includeidoxuridine, acyclovir (acycloguansoine), ganiciclovir, adensosine arabinoside (AraA, Vidarabine), Ara-AMP AraC (cytarabine), Ara-CMP, azidothymidine (AZT), ribavirin, didenosine (DDI), dideoxycytosine (DDC), stavudine (d4T), Epivir (3TC), abacavir (ABC), iododeozyuridine (DU), Valacyclovir, and bromovinyl deoxiuridine (BVDU).

Nucleoside analogs that include sugar modifications are acyclovir (a guanosine analogue), ganiciclovir (a 2′-deoxyguanosine analogue, similar to acyclovir but with an extra hydroymethyl group on its side chain), Valacyclovir is the hydrochloride salt of l-valyl ester of acyclovir, AraA, DDI, DDC. Nucleoside analogues with base modifications include DU, BVDU, and Ribavirin which is a guanosine analogue.

In one embodiment, the anti-viral nucleoside analogues can be any 2,3-didexoxynucleoside analogues or 2,3 nucleoside analogue; oxathiolanyl 2,3-dideoxynucleoside or oxathiolanyl 2,3-nucleoside analogue; dioxolanyl 2,3-dideoxynucleoside analogue or dioxoanyle 2,3-nucleoside analogue; carbocyclic 2,3 -dideoxynucleoside analogue or carboxyclic 2,3-nucleoside analogues; an acyclic nucleoside analogue; a prodrug, such as an ester or phophlipid prodrug, dihyrdopyridine or pronucleotide and dinucleotide analogue, acetylated nucleoside analogues In another aspect, anti-viral nucleoside analogues that can be used in the conjugates of the present invention are well known in the art and include but are not limited to: AraA, AraC and guanosine analogues such as ribavirin and pharmaceutically acceptable salts thereof.

For instance, guanosine analogues that can be used are those described in U.S. Pat. Nos. 6,063,772 or 4,950,647; A. R. Karlstrom et al. Antimicrob Agents Chemother. 1986 January, 29 (1):171-174.

For instance, AraA analogues that can be used, can be for instance, those described in U.S. Pat. No. 6,582,947; Canadian patent application numbers 2,362,805; 2,231,442;; 2,322,487; and 2,231,442.

In one embodiment, the AraC analogues can be those described in 2,180,348; 2,362,805; 2,322,487; or 2,431,839.

In one aspect the analogues of the present invention are capable of binding to hemoglobin either directly or through a linker.

Ribavirin

Ribavirin is a nucleoside analogue and has been used in anti-viral therapy and as an immunomodulator. The present inventors are the first to determine that delivery of ribavirin directly to macrophages enhances its activity. They were also the first to deliver ribavirin to hemoglobin-haptoglobin receptor bearing cells, such as hepatocytes, and to CD163 receptor bearing cells, such as macrophagesand have shown that this unexpectedly enhances the effectiveness of ribavirin therapy As such the present invention provides ribavirin and ribavirin conjugates, and ribavirin compositions, and methods of using same in ribavirin therapy, immunomodulation therapy, anemia, anti-viral therapy, modulation of cytokine secretion, modulation of macrophages, treatment of a macrophage disorder or other disorder in which ribavirin has been found to be useful in the treatment of.

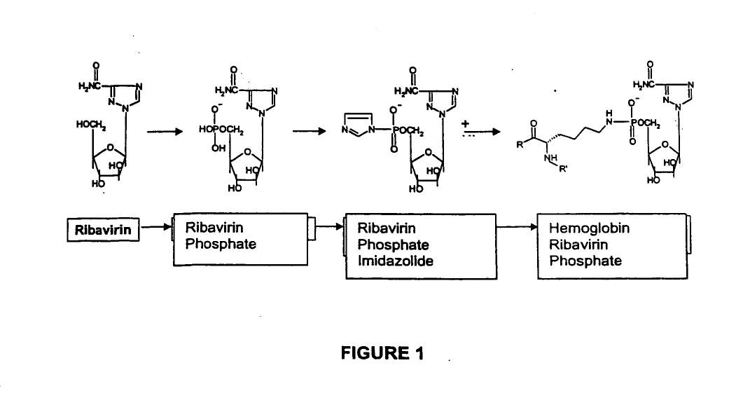

Ribavirin that can be used in the present invention can be but are not limited to: ribavirin as disclosed in FIG. 1 or shown below (I) or a chemical equivalent or obvious chemical equivalent thereof, such as an analogue or derivative or homolog thereof or a pharmaceutically acceptable salt thereof .

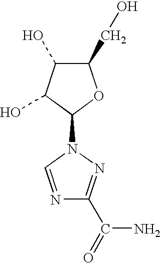

A chemical equivalent of ribavirin would be one that is analogue, derivative, isomer, homolog, or other modified version of ribavirin that has the desired equivalent function, whether it be anti-viral function or other desired function of ribavirin.

Modified ribavirin can be used, that is modified at the sugar and/or base moiety as long as it has the desired functions. And is capable of binding hemoglobin.

For instance, modified ribose moieties and their phosphorylated versions, such as: 2′-deoxy ribavirin; 3′-deoxy ribavirin; 2′, 3′-dideoxy ribavirin; 2′, 3′-epoxy ribavrin; 2′, 3′-dideoxy-2′, 3′-dehydro ribavarin; 5′-nor-carbocyclic ribavirin; Levovirin, the L-enantiomer of ribavirin (D).

In another embodiment, modified base moieties and their phosphorylated versions can be used, such as: Viramidine, previously known as Ribamidine, the carboxamidine prodrug of carboxamide ribavirin; activated by adenosine deaminase; pyrazole nucleoside analogues as bioisosteres of triazole ribavirin, also called dideaza analogues In yet another embodiment, analogues that differ in the degree of phosphorylation of ribavirin can be used; e.g., mono, di, and tri 5′, 2′, and 3′, or combinations of all three.

Ribavirin-like compounds that can be used in the invention are those compounds that are nucleoside analogues, such as other guanosine analogues and have anti-viral activity or other equivalent activity to ribavirin.

In another embodiment, the ribavirin nucleoside analogues described in Canadian application numbers 2,384,326; 2,246,162; 2,236,344; 2,278,158, 2,213489;Hoffman et al, 1973, Antimicrob. Agents Chemother. 3:235 or Sidwell et al. 1972, Science 177:205; or U.S. Pat. No., 4,328,336; 3,803,126 could also be used.

The use of the term a “ribavirin” herein includes the ribavirin, ribivirin-like compounds, analogues or derivatives or ribvarin, chemical equivalents or obvious chemical equivalents thereof or their pharmaceutical acceptable salts. All of these can be used in the invention as long as they are capable of conjugating to hemoglobin. In one embodiment, they bind to hemoglobin (directly or indirectly through a linker or the like), in a manner that enables the conjugate to bind to haptoglobin.

In so far as they are applicatble, the modifications and analogues described for Ribavirin, AraA, or AraC can be applied to other anti-viral nucleoside analogues.

Hemoglobin-Anti-Viral Nucleoside Conjugates, Such As Hemoglobin-Ribavirin Conjugates and AraA and AraC Conjugates

The present invention provides a synthetic hemoglobin-anti-viral nucleoside conjugate, such as a hemoglobin-ribavirin conjugate designed to deliver the nucleoside to cells bearing receptors for the hemoglobin and its derivatives-, such as cells bearing hemoglobin-haptoglobin receptors such as hepatocytes, CD163 bearing cells and macrophages. Selective uptake of haptoglobin-hemoglobin-ribavirin has been herein demonstrated in vitro in hepatic cells and cells expressing CD163, and enhanced effect of haptoglobin-hemoglobin-ribavirin vs. free ribavirin has been demonstrated in vitro in macrophages and in vivo in virus-infected mice. Similar selective in vivo uptake of AraA and AraC conjugates by CD163 bearing cells has also been demonstrated in the examples. In another embodiment, the invention also provides a hemoglobin- anti-viral nucleoside conjugates capable of binding haptoglobin or that comprises haptoglobin. It avoids the systemic toxicity associated with chronic nucleoside therapy such as ribavirin therapy. The conjugate of the present invention can achieve greater efficacy of nucleoside analogue therapy, such as ribavirin therapy. With regard to hemoglobin-ribavirin conjugates, it also is effective in maintaining optimal ribavirin levels in patients, such as HCV patients, who would otherwise require dose reduction or discontinuation of ribavirin therapy. Acid phosphatase, a lysosomal enzyme, has been shown to release bioactive ribavirin from hemoglobin-ribavirin in vitro. Similar release of active drug is expected following lysosomal uptake of the conjugate in target cells.

The hemoglobin-anti-viral nucleoside conjugate, such as the ribavirin conjugate, can in one embodiment be formed via the reaction scheme shown in FIG. 1 or outlined in Example 2. But the present invention is not intended to be limited to said reaction scheme or mode of conjugation. A person skilled in the art would appreciate that other modes of conjugation could be used. In another embodiment, the hemoglobin can be any hemoglobin as previously described herein. In another embodiment, the ribavirin can be a ribavirin or ribavirin analogue as previously described herein. Conjugation of AraA and AraC is described in Example 4.

In one embodiment, the present invention provides hemoglobin-anti-viral nucleoside analogues that are hemoglobin phosphoramidate anti-viral nucleoside analogues. The bond may enable cleavage of the nucleoside analogue in a phosphorylated form. In one embodiment this is the active form of the anti-viral nucleoside analogue.

In one embodiment, the molar ratio of Hb: anti-viral nucleoside analogue is 1:5-1:20. In one embodiment the molar ratio is 1:5-1:15.

In one embodiment, the molar ratio of Hb:ribavirin is about 1:5 to about 1:10. In another embodiment, the molar ratio is about 1.8 (Hb:ribavirin).

Pharmaceutical Compositions

The present invention provides pharmaceutical compositions comprising the hemoglobin-anti-viral nucleoside analogues of the present invention. In one aspect the pharmaceutical compositions of the invention can target delivery of anti-viral nucleoside analogues such as Ara A, AraC and ribavirin to macrophages. It also provides pharmaceutical compositions that can deliver the anti-viral nucleoside analogues, such as AraA, AraC and ribavirin to hemoglobin or hemoglobin-haptoglobin receptor bearing cells and to CD163 receptor bearing cells. The compositions may be administered to living organisms including humans, and animals. In another embodiment, the invention also provides hemoglobin-drug conjugates for delivery of the drug to macrophages.

The pharmaceutical composition may be administered in a convenient manner such as by direct application to the infected site, e.g. by injection (subcutaneous, intravenous, etc.). In case of respiratory infections, such as SARS, it may be desirable to administer the conjugates, such as the ribavirin compositions of the present invention directly to the lungs, through known techniques in the art. Depending on the route of administration (e.g. injection, oral or inhalation, although injection is a preferred mode of administration), the pharmaceutical compositions may be coated in a material to protect the compound from the action of enzymes, acids and other natural conditions that may inactivate the compound.

The compositions described herein can be prepared by per se known methods for the preparation of pharmaceutically acceptable compositions which can be administered to subjects, such that an effective quantity of the active substance, (e.g. ribavirin) is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985) or Handbook of Pharmaceutical Additives (compiled by Michael and Irene Ash, Gower Publishing Limited, Aldershot, England (1995)). On this basis, the compositions include, albeit not exclusively, solutions of the substances in association with one or more pharmaceutically acceptable vehicles or diluents, and may be contained in buffered solutions with a suitable pH and/or be iso-osmotic with physiological fluids. In this regard, reference can be made to U.S. Pat. No. 5,843,456. As will also be appreciated by those skilled, administration of substances described herein may be by an inactive viral carrier.

In addition to pharmaceutical compositions, compositions for non-pharmaceutical purposes are also included within the scope of the present invention. In such instances, the carrier can be selected to deliver anti-viral nucleoside analogues, such as araA, araC and ribavirin to macrophages in vitro or other suitable receptor bearing sites to be used as a diagnostic or research tool. The anti-viral nucleoside analogue, such as araA, araC and ribavirin can be labelled with labels known in the art, such as florescent labels or the like.

Applications

Administration of a therapeutically effective amount, “effective amount” or “sufficient amount” of pharmaceutical compositions of the present invention is defined as an amount effective, at dosages and for periods of time necessary to achieve the desired result, including clinical results, and, as such, an “effective amount” depends upon the context in which it is being applied. For example, a therapeutically active amount of a substance may vary according to factors such as the disease state, age, sex, and weight of the individual, mode and form of administration and the ability of the substance to elicit a desired response in the individual Dosage regimes may be adjusted to provide the optimum therapeutic response. For example, several divided doses may be administered daily or the dose may be proportionally reduced as indicated by the exigencies of the therapeutic situation. For example, in the context of administering an effective amount of an anti-viral nucleoside analogue, such as ribavirin, ribavirin conjugate or hemoglobin-ribavirin conjugate of the present invention is an amount sufficient to achieve such desired activity, e.g. anti-viral, and/or macrophage modulator; and/or cytokine modulator; and/or immunomodulator, and/or inflammatory response modulator.

In one embodiment, the effective amount is based on plasma concentration of the nucleoside. In another embodiment is based on dosage per day or dosage per kg of body weight. The desired amount can depend on desired use or mode of administration.

As used herein, and as well understood in the art, “treatment” is an approach for obtaining beneficial or desired results, including clinical results- Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “modulate” as used herein includes the inhibition or suppression of a function or activity (such as ribavirin activity, AraA, AraC or macrophage activity) as well as the enhancement of a function or activity.

The term “animal” as used herein includes all members of the animal kingdom including human that can benefit from treatment with the drug conjugates of the present invention, for instance animals that have or are carriers of viral infections, such as birds, mammals (such as horses, pigs, dogs, cats, humans). In one embodiment, the animal is a human.

The phrase, “Subject in need thereof” as used herein means any animal that has or is suspected of having a condition that can be treated or prevented by the hemoglobin-anti-viral nucleoside analogues of the present invention.

The invention provides methods and uses of a drug delivery system for targeted anti-viral nucleoside analogue delivery to cells having hemoglobin, CD163 or hemoglobin-haptoglobin receptors. In one embodiment, the invention provides a method of targeted drug delivery to macrophages. The invention further provides a ribavirin drug delivery system and compositions and conjugates to target macrophages to modulate the immune and inflammatory response, preferably to enhance the anti-viral and/or immunomodulatory and/or anti-inflammatory activity of ribavirin by targeting them to macrophages The conjugates of the present invention also increase the efficacy of ribavirin due to increased ribavirin analogue bioavailability, half-life and stability. It also reduces systemic toxicity by targeting ribavirin analogues to macrophages. In one embodiment, the ribavirin analogues can be delivered using carriers known to target and deliver drugs and other substances to macrophages, including analogues or derivatives of hemoglobin that are capable of binding haptoglobin and that bind the hemoglobin or hemoglobin- haptoglobin receptors. The ribavirin analogue can be conjugated to hemoglobin or to hemogobin derivatives in a manner that enables binding to a suitable receptor, such as CD163, said receptor being known to be present on macrophages and hepatocytes and can target ribavirin-like compound delivery to these sites, The ribavirin drug delivery system and compositions and conjugates of the present invention can be used to control or treat conditions related to immune /inflammatory response, or other responses mediated by macrophages. Although, ribavirin is used herein to exemplify the embodiments of the invention, the present invention also provides similar drug delivery systems for other anti-viral nucleoside analogues, such as AraA and AraC.

Also, hemoglobin can be used to deliver a wide range of drugs or substances to macrophages Said drugs or conjugates are not necessarily limited to a ribavirin, but can be other anti-virals, anti-viral nucleoside analogues or anti-bacterial or non-anti-viral drugs or substances.

The anti-viral nucleoside analogue compositions and/or conjugates and/or complexes of the present invention can be used in the treatment of a number of conditions, or in the preparation of a medicament for the treatment of a number of conditions, such as a CD163 cell or macrophage mediated condition. It can enhance the treatment of viral infections. For instance, ribavirin compositions, conjugates and complexes of the present invention can be used in the treatment of coronaviruses (e.g., MHV-3, SARS), hepatitis C, RSV, Lassa Fever and the like, by administration to a subject in need thereof.

The hemoglobin conjugates of the present invention can be used to target delivery of a substance to macrophages for the treatment of a number of conditions, such as tuberculosis, anthrax, or other conditions known to be mediated through macrophages.

The anti-viral nucleoside compositions, conjugates and complexes of the present invention can also be used to reduce or alleviate anemia, such as hemolytic 15 anemia, often associated with nucleoside analog treatment- In one embodiment, the compositions, conjugates and complexes can be used to enhance erythropoiesis (see, PCT/CA97/00601, filed Aug. 27, 1997).

The anti-viral nucleoside analogue compositions, conjugates and complexes of the present invention can also be used as diagnostic and/or research tools, for instance in the investigation of macrophage mediated responses, inflammatory responses, immune responses and the like. This can be done using a labeled anti-viral-nucleoside analogue, such as ribavirin, AraA, AraC, or conjugates with hemoglobin or conjugate-complexes of the invention. Suitable labels are well known in the art, such as fluorescent or radio-labels. The labeled anti-viral-nucleoside analogue, hemoglobin conjugates or complexes of the invention can be administered to a macrophage or cell comprising CD163 under conditions that prevent or enable conjugate/cell interaction in the presence of a potential modulator and monitoring the effect of said potential modulator on the effect of the conjugate on said cell compared to a control. Such a control can be an internal or external control. It can be a parallel experiment in the presence or absence of hemoglobin, the anti-viral-nucleoside conjugate or complex, the anti-viral nucleoside or any combination. A person skilled in the art would be able to develop a suitable control for what is to be studied. In this regard one could monitor viral load, cytokine levels or other indicator. In this way, one can identify potential co-therapy compounds, or mechanisms of action for further evaluation, treatment or drug development.

The present invention shall now be illustrated by the following examples Such examples are for illustrative purposes only and are not intended to limit the scope of the present invention or appended claims.

### EXAMPLES

### Example 1—Preparation of Hemoglobin

Stroma free hemoglobin was prepared using techniques known in the art. In the present instance, human hemoglobin was obtained from outdated red blood cells, and purified by the displacement chromatography process described in U.S. Pat. No. 5,439,591 (Pliura et al.). Non-intramolecularly cross-linked hemoglobin was used for the Examples below.

### Example 2—Preparation of Hemoglobin-Ribavirin Conjugate Synthesis of Ribavirin Phosphate Imidazolide

Ribavirin phosphate was synthesized by derivatisation of ribavirin at its primary hydroxyl group using phosphooxychloride and dimethylphosphate (Allen, et al., J Med Chem. 1978 August;21(8):742-6.), and monitored for ribavirin modification by C18 reverse-phase HPLC. Following completion of the reaction, the ribavirin phosphate (1 mmol) was mixed with 10 g of fine charcoal (100-400 mesh). The charcoal-reaction mixture was centrifuged at 2000 g for 15 min and the supernatant recovered. The wash steps were repeated until no inorganic phosphate could be detected in the supernatant as assayed using the Ames method (Ames BN (1966), Assay of inorganic phosphate, total phosphate and phosphatases. Methods Enzymol 8: 115-118). The charcoal was extracted with EtOHtwater/NH₄OH (10:10:1) and the pooled extract evaporated to dryness The resulting ribavirin phosphate ammonium salt was converted to its free acid (Streeter et al, Proc. Natli Acad. Sci. USA 1973 April; 70(4):1174-8). Purity of the ribavirin phosphate was evaluated using 2 assays. Acid phosphatase was used for complete enzymatic cleavage of ribavirin from ribavirin phosphate, followed by quantification of the released ribavirin by C18 reverse-phase HPLC. C18 reverse-phase HPLC was performed on a C18 Phenomenex Luna column (4.6×250) using isocratic elution with water/TFA 0.1% pH 2.9, flow 1 ml/min. Total inorganic phosphate content of ribavirin phosphate was measured using the Ames method (described below). Purified ribavirin phosphate was converted to its imidazolide (Fiume et al., Anal Biochem. 1993 August 1:212(2):407-11), with slight modifications. The reaction was performed under dry N₂, using dry solvents. Typically, 324 mg (1 mmol) of ribavirin phosphate was dissolved in 10 ml anhydrous DMF. 5 mmol carbonyidiimidazole in 5 ml DMF was added, followed by 5 mmol imidazole in 5 ml DMF. The solution was stirred at RT for 30 min and the DMF evaporated. The remaining oil/solid was dissolved in 2 ml anhydrous EtOH, followed by precipitation with the addition of 20 ml anhydrous ether. The precipitate was washed twice with ether, and residual ether was removed with a gentle stream of dry N₂. The ribavirin phosphate imidazolide was used immediately for conjugation to Hb.

Conjugation of Ribavirin Phosphate Imidazolide With Hb.

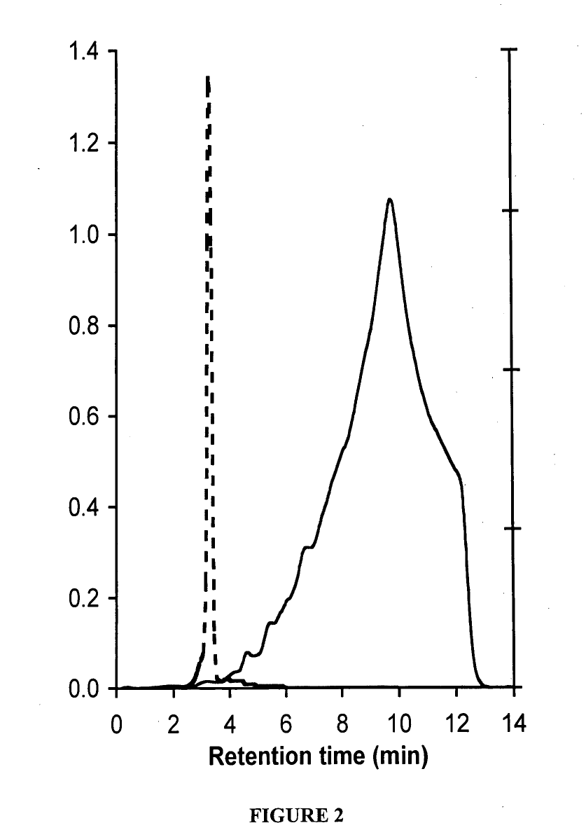

0.06 μmol of Hb (CO form) was mixed with 6.6 μmol ribavirin phosphate imidazolide at a final concentration of 0.8 μM in 0.1 M NaHCO₃/Na₂CO₃, pH 9.5. The pH of the reaction mixture was monitored over the first hr, and maintained at pH 9.5-9.6 by addition of 0.2 M Na₂CO₃. After the pH of the reaction had stabilized, the reaction mixture was charged with CO for 15 min and the reaction allowed to continue under CO at 37° C. for 96 hr. Hb was monitored for drug modification using anion-exchange chromatography Anion exchange chromatography was performed on a Poros H/HQ (4.6/100) column, using a pH gradient 8.3-6.3 over 10 min (mobile phase 25 mM Tris pH 8.3, 25 mM bisTris pH 6.3) with a flow rate of 4 ml/min. All Hb was modified as evidenced by later elution on anion exchange media relative to unmodified Hb control, due to the added net negative charge resulting from modification of lysine side chain amino groups with the phosphate containing conjugant (FIG. 2). The conjugate was dialysed (MWCO 10 kDa) against Ringer's Lactate (3×0.5 L), sterile filtered (0.2 μm) and charged with CO prior to storage at −80° C.

Determination of Molar Drug Ratio.

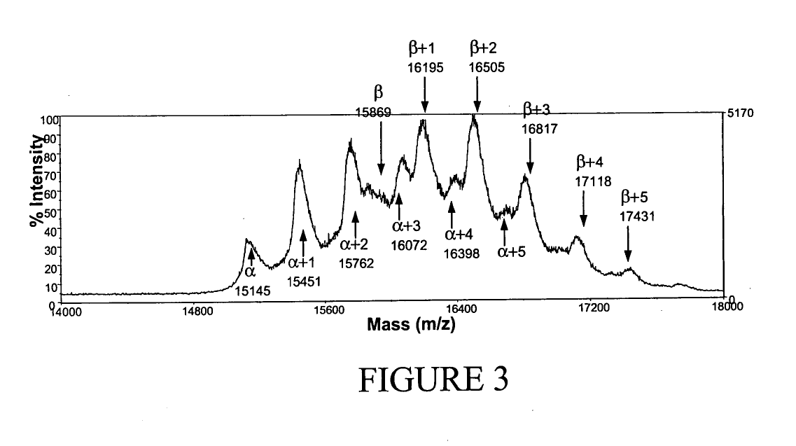

The molar drug ratio of Hb-ribavirin conjugate was determined by quantification of ribavirin released by enzymatic cleavage using the acid phosphatase assay and determination of total inorganic phosphate using the inorganic phosphate assay. The molar concentration of Hb protein was determined using the Drabkins assay kit for Hb (Sigma). MALDI-TOF mass spectrometric analysis of the Hb-ribavirin conjugate indicated up to at least 5 ribavirin phosphate groups attached to both alpha and beta chains of the Hb (FIG. 3). However, on average 8 ribavirin molecules covalently linked to each hemoglobin molecule. For the acid phosphatase assay, 5 nmol (0.3 mg) of the Hb-ribavirin conjugate was diluted into 0.3 ml of 1 mM NaOAc/HAc buffer, pH 4.8. 3 units of a freshly prepared acid phosphatase (Type IV-S, potato) was added, and the enzymatic reaction allowed to proceed at 37° C. for 2 hr. Hb precipitate was removed by centrifugation and the supernatant analysed for ribavirin by C18 reverse-phase HPLC. For evaluation of biological activity of released ribavirin, the supernatant was dialysed against PBS and concentrated prior to analysis. For the inorganic phosphate assay, total inorganic phosphate was determined using the method of Ames, 1966. The volume of 10% Mg(NO₃)₂ in EtOH was optimized to 80 μl for assay of 5 nmol Hb-ribavirin conjugate.

HP Binding Assay.

To determine retention of Hp binding by Hb-ribavirin conjugate, a complex was allowed to form with a 10% molar excess of human Hp, at RT for a minimum of 30 min. Size exclusion HPLC (SEC) analysis indicated formation of a higher MW complex corresponding to the Hp complex of the Hb-ribavirin. SEC was performed using a Pharmacia Superdex 200 column using 0.5M MgCl2/25 mM Tris, pH 7.2 at a flow rate of 0.4 ml/min, with detection at 414 nm. Formation of the complex of conjugate with Hp was confirmed by elution of Hb-containing species that appeared as peaks eluting earlier than the non-complexed Hb-drug conjugates.

Preparation Of The Fluorescein-Hb-Ribavirin Double Conjugate.

A 1 mM solution of fluorescein maleimide (Pierce 46130) was prepared in PBS. Hemoglobin or hemoglobin-ribavirin conjugate was added to a concentration of 100 μM, and incubated 4 hr in the dark at RT with gentle agitation. The sample was then dialyzed extensively against PBS to remove any unbound fluor. Based on the concentration of fluor and protein in the purified conjugate, determined by fluorimetry and Coomassie analysis, respectively, the ratio of fluor label to Hb-RV was approximately 1. RP HPLC analysis coupled with fluorescence detection showed all fluorescence to be associated with the β-chain, indicating the expected attachment of the fluorescein maleimide to the surface reactive βCys93 thiol group. Binding of the fluorescein labelled Hb-RV to Hp was also verified by size exlusion HPLC analysis. Binding to Hp was verified the a shifting of the Hb derivative peaks to an earlier retention time corresponding to an increase in molecular weight upon formation of the Hp-Hb(FI)-RV complex.

In vitro Bioactivity Assay of Ribavirin From Hb-Ribavirin Conjugate.

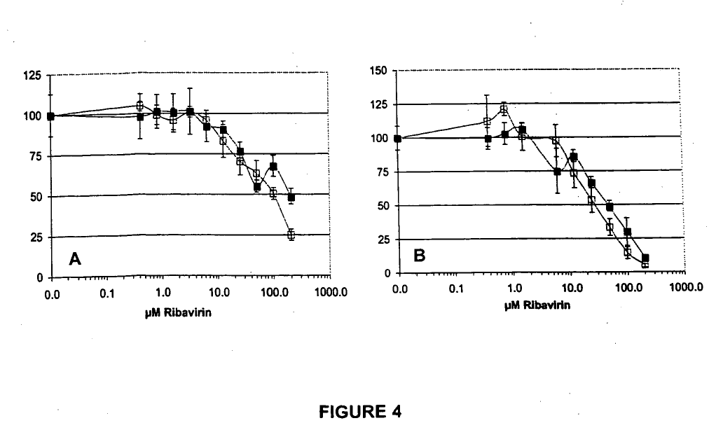

Ribavirin recovered from acid phosphatase cleavage of Hb-ribavirin conjugate was evaluated for bioactivity in an in vitro cell proliferation assay using the Cell Proliferation ELISA Bromodeoxyuridine (BrdU) kit (Roche, Cat. No. 1 647 229). Human hepatoma HepG2 cells and mouse hepatocyte AML12 cells were plated at a density of 4×10⁴ cells/well, and 1×10⁴ cells/well, respectively, in flat bottom 96-well plates. The cells were allowed to grow for 24 hours, at which time they were treated in quadruplicate with ribavirin or ribavirin from Hb-ribavirin conjugate for 6 hours. The treatments were removed, and fresh media containing BrdU was added to the wells and the incubation continued for 18 hr. The standard BrdU ELISA assay was then followed according to the kit protocol. Cleaved ribavirin activity was equivalent to unmodified ribavirin control (FIG. 4), demonstrating the ribavirin is not detrimentally altered by the conjugation and cleavage processes, and suggesting that activity of ribavirin cleaved from the conjugate in vivo will have activity similar to free ribavirin.

Internalization Assay.

To evaluate uptake of Hb-ribavirin conjugate by hepatic cells, internalization assays were performed using fluorescein-tagged Hb and Hb-ribavirin conjugate (Zuwala-Jagiello and Osada, 1998, “Internalization study using EDTA-prepared hepotocytes for receptor-mediated endocytosis of haemoglobin-haptoglobin complex”; The International Journal of Biochemistry & Cell Biology; England, August 1998, vol. 30, No. 8; pp. 923-931, XP00107508).

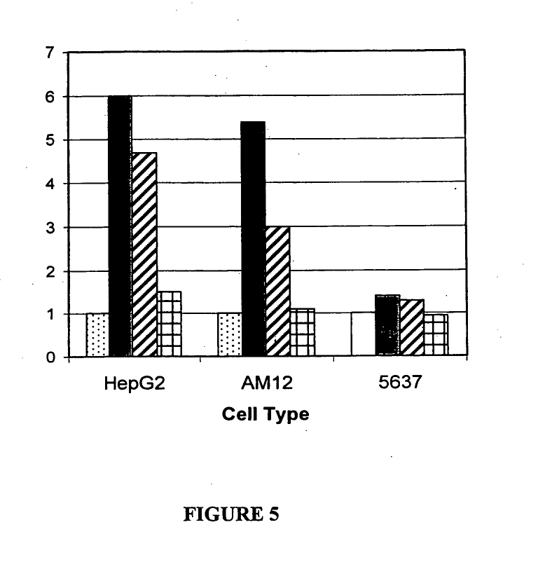

HepG2 cells and 5637 bladder carcinoma cells were plated in 12-well plates at 2.5×10⁶ cells/well, and mouse AM12 hepatocytes at 2×10⁵cells/well. The cells were allowed to grow for 48 hours. Media was removed and the cells were washed with HBSS containing 2 mg/ml BSA (HBSS/BSA). Hb or Hb-ribavirin conjugate labelled with fluorescein was complexed with Hp (1:1 molar ratio) in HBSS/BSA, and added to cells to a final concentration of 500 μg/ml. The labelled complexes were allowed to bind for 2 hr at 4° C. ATP was added to 1 mM and receptor-mediated internalization was initiated by incubation at 37° C. for various times. The cells were washed with HBSS/BSA and surface-bound ligands were stripped from cells by incubation in 0.2 M acetic acid/0.5 M NaCl for 10 minutes. The cells were washed with PBS and lysed with 2 M NaOH. The solubilized cell extract was transferred to a flat bottom 96 well plate, and fluorescence measured (485 nm excitation/530 nm emission) using a fluorometric plate reader (Packard Fluorocount). Both the Hb and Hb-ribavirin, complexed to Hp, were taken up by the liver derived cell lines (HepG2 and mouse AM12) and neither was effectively internalized by the non-liver cell line (5637 bladder carcinoma), demonstrating the selective targeting of the Hb-ribavirin complex to cells bearing receptors the Hb-Hp complex (FIG. 5). A labelled albumin control was not significantly internalized by any of the cell lines, thereby confirming that the level of Hb uptake in the liver cell lines was not due to passive transport of macromolecules.

### Example 3—In vitro and In vivo Studies Of Hemoglobin-Ribavirin Conjugates

In The Treatment of MHV-3

The drug delivery effects of free ribavirin and hemoglobin-ribavirin conjugate (Hb-ribavirin), prepared as in Example 2 and complexed to haptoglobin, were compared in mice infected with murine hepatitis virus strain 3 (MHV-3), a coronavirus that produces fulminant hepatitis in mice. The molar ratio of conjugated ribavirin to hemoglobin was approximately 8:1.

Methods.

These studies were designed to examine the potential for haptoglobin-hemoglobin-ribavirin (Hp-Hb-Ribavirin) to protect against MHV-3 infection in vivo and to assess the anti-viral and anti-inflammatory effects in cultures of macrophages in vitro.

In vivo

| | |
|---|---|
| Day −1 | Treatment (All infusions were 100 μl in PBS) |
| | 1) PBS (n = 5) |
| | 2) Hp-Hb-Ribavirin (6 mg RV/kg/ay, n = 10) |
| | 3) Ribavirin (18 mg RV/kg/day, n = 10) |
| Day 0 | Infection (i.p. 100 pfu MHV-3 in PBS) + Treatment |
| | (1, 2, 3) |
| Days 2-5 | Daily: |
| | Measure survival |
| | Sacrifice 2 mice per group for measures of: |
| | Serum ALT |
| | Hemoglobin |
| | Hematocrit |
| | Liver viral titre |
| | Liver histopathology + |
| | Treatment of remainder (1, 2, 3), excluding Day 5 |
| | (end of study) |

Mice were divided into 3 treatment groups as follows:

Group 1:Mice infected with MHV-3 and treated with PBS as controls (n=5)

Group 2:Mice infected with MHV-3 and treated with Hp-Hb-ribavirin at 6 mg conjugated ribavirin/kg/day (n=10)

Group 3:Mice infected with MHV-3 and treated with ribavirin at 18 mg/kg/day (n=10)

All groups of mice were infected with 100 plaque forming units (PFU) of MHV-3 by intraperitoneal injection. Treatments were given daily by intravenous tail vein injection starting at day −1 and continuing to the end of the experiment. Blood samples were collected daily, prior to daily test article infusion, and analyzed for evidence of hepatitis by liver biochemistry (alanine aminotransferase, bilirubin) disturbances in hematologic parameters (hemoglobin, white blood cell count, platelet count), renal dysfunction (creatinine, blood urea nitrogen). Liver tissues were collected, fixed in formalin and examined by routine histology (hematoxylin and eosin) for hepatic necrosis and by immunohistochemistry for fibrin deposition and necrosis. Viral titers were determined by cytopathic effect assay using snap frozen liver tissue.

In Vitro

Macrophages were isolated from mice after injection of intraperitoneal thioglycollate. Macrophages were pretreated with free ribavirin (200 ug/ml) or Hp-Hb-ribavirin (1 mg/ml containing approximately 10 ug conjugated ribavirin/ml) one hr prior to infection with 1000 PFU MHV-3 (m.o.i. 10⁻³). At intervals, macrophages were harvested and analyzed for viral titers and production of inflammatory mediators, tumor necrosis factor-α (TNF-α) and interferon-γ (IFN-γ). Results.

Clinical Behaviour

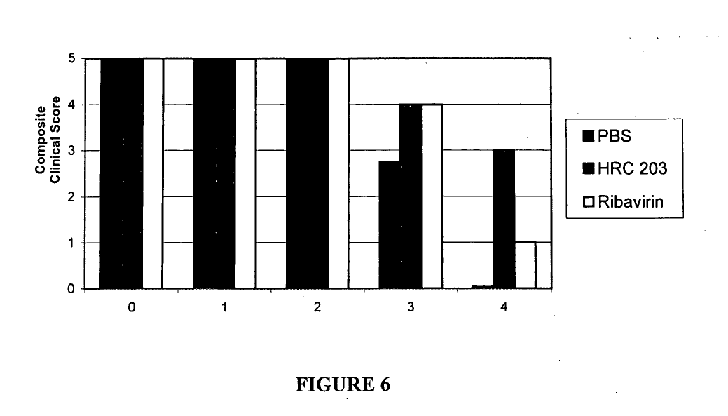

Mice treated with the hemoglobin-ribavirin conjugate of the invention exhibited superior clinical behaviour after infection with MHV-3. The infected controls (PBS/MHV-3 alone) at day 2-4 had ruffled fur, were shaking and were inactive. The infected mice treated with ribavirin alone had ruffled fur and were lethargic at day 2-5. However, the infected mice treated with the hemoglobin-ribavirin conjugate of the invention exhibited superior clinical behaviour (i.e., were active, had normal respiration and fur texture and no shaking) and behaved like uninfected normal mice. A graph of the composite clinical score versus days post-infection for the three test groups is illustrated in FIG. 6.

Anemia

MHV-3 infected mice treated with hemoglobin-ribavirin do not develop anemia that is normally caused by ribavirin therapy. As stated previously, prior art reports that ribavirin therapy causes does-limiting hemolytic anemia. In the present study, the ribavirin group showed decreased hematocrit and hemoglobin below the normal range consistent with anemia over the 3 days following infection, while the hemoglobin-ribavirin treated animals stayed in the normal range (See Table 1). On day 5 it was noted that the ribavirin treated animal were dehydrated as compared to the hemoglobin-ribavirin treated animals which results in elevating their hematocrit and hemoglobin levels.

Liver Necrosis and Fibrin Deposition

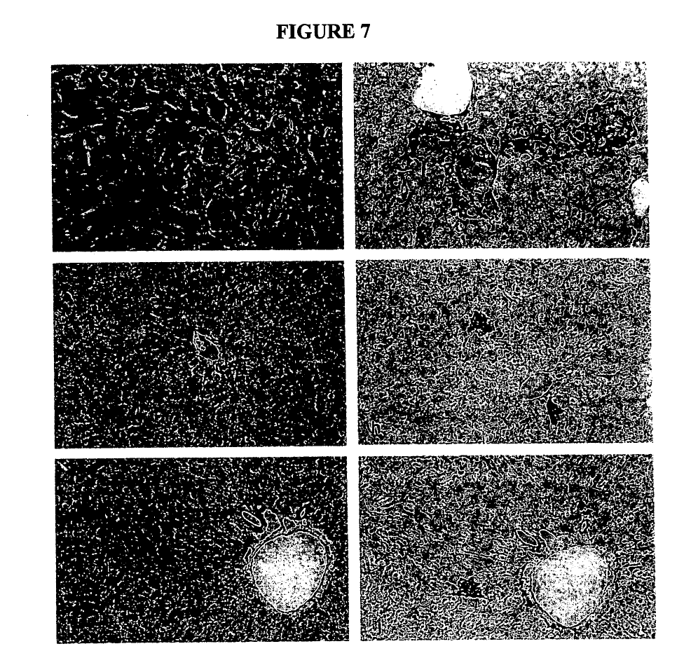

Histopathology results indicated that treatment of MHV-3 infected mice with Hp-Hb-Ribavirin resulted in delaying and reducing the course of liver necrosis and fibrin deposition caused by MHV-3 infection as compared to untreated controls. Reduction in liver necrosis was similar between the Hp-Hb-Ribavirin and free ribavirin groups, despite the fact that the dose of conjugated ribavirin in the Hp-Hb-Ribavirin was only ⅓ of that of free ribavirin used. The results at day 3 are illustrated in FIG. 7 and described below.

Day 3 Liver: PBS Control (FIG. 7 e, f) (350× magnifcation)

There are marked diffuse hepatic cellular changes with multiple areas of confluent hepatocellular necrosis throughout the liver. Approximately 60% of the liver is necrotic. The immunostain shows striking fibrin deposits that match the areas of necrosis; the fibrin is deposited especially in sinusoids within and around the areas of necrosis. This is the classical hepatic pathology of MHV-3 induced murine fulminant viral hepatitis. The natural progression is rapid extension of the necrosis to involve the entire liver once it has reached this stage. It is graded here as 3+ out of 4.

Day 3 Liver: Hp-Hb-Ribavirin-treated group (FIG. 7 g, h) (250× magnification)

The liver is characterized by widely scattered microfoci of liver cell necrosis. The lesions are very discreet. The immunostain shows sharp localization of fibrin in sinusoids in the areas of necrosis. This is early hepatic necrosis in MHV-3 viral hepatitis. Hepatic changes are variable. The extent of the necrosis is graded as 1+ and is estimated as 5-10%.

Day 3 Liver: Ribavifin-treated group (FIG. 7 i, j) (250× magnification)

There are widely scattered microfoci of necrosis. The changes are similar in type and extent to those shown in g and h of day 3 Hp-Hb-Ribavirin-treated liver.

Survival

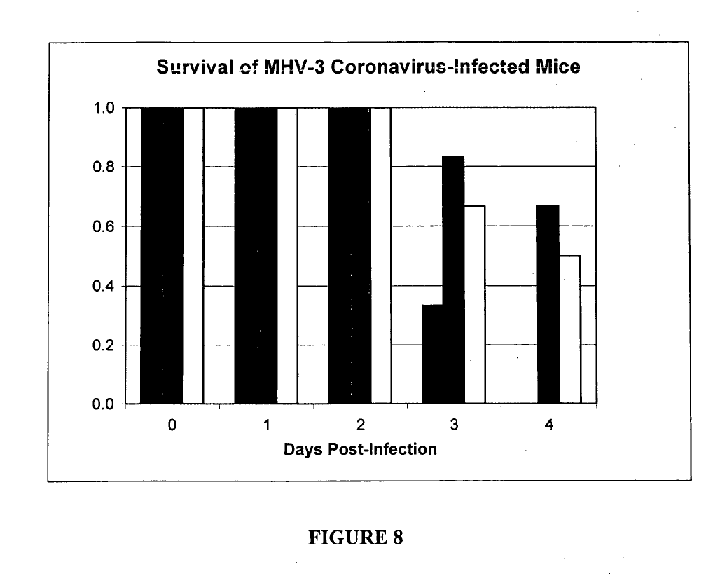

Animals were sacrificed daily post infection to recover tissues for analysis. The number of animals remaining was monitored for survival, and the results as presented include death both from disease and sacrifice. The fraction surviving is calculated based on animals surviving at the beginning of each day at the time of sacrifice, and do not include animals which die by the end of that day. Survival of MHV-3 infected mice treated with hemoglobin-ribavirin exceeded that of free ribavirin despite the fact that the dose of ribavirin in the Hb-conjugate was ⅓ of that of free ribavirin. Results are illustrated in FIG. 8.

Anti-viral Activity in Vivo and in Vitro

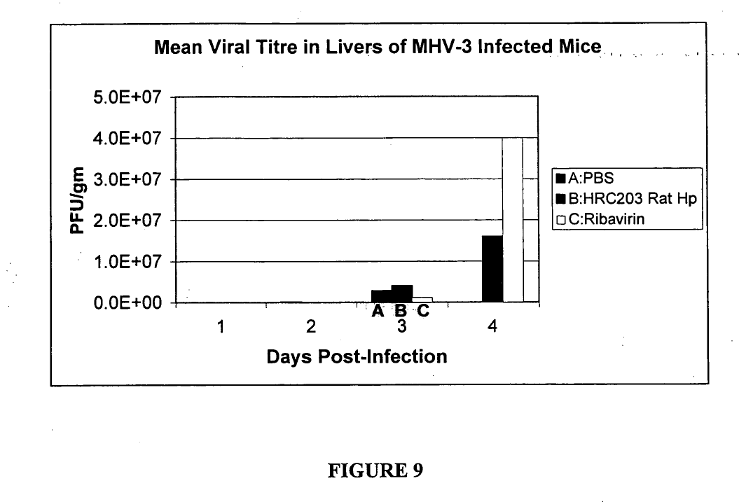

Livers harvested from MHV-3 infected mice treated with Hp-Hb-RV demonstrated a significantly lower viral titer than mice treated with free ribavirin alone. Results are illustrated in FIG. 9.

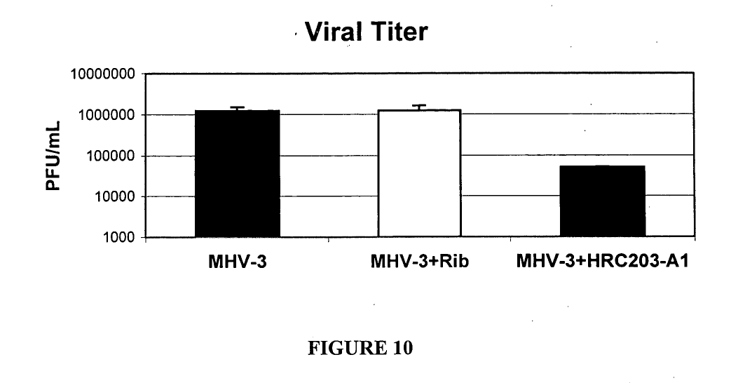

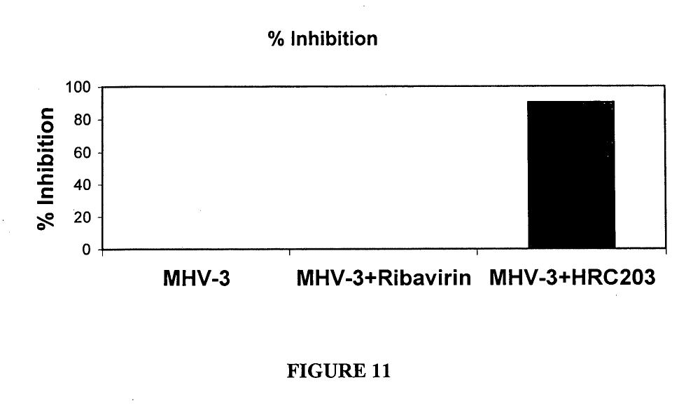

Macrophages treated with Hp-Hb-RV in vitro had a marked reduction in MHV-3 viral titers (FIG. 10) and showed greater inhibition of viral replication (FIG. 11) in contrast to macrophages treated with free ribavirin alone.

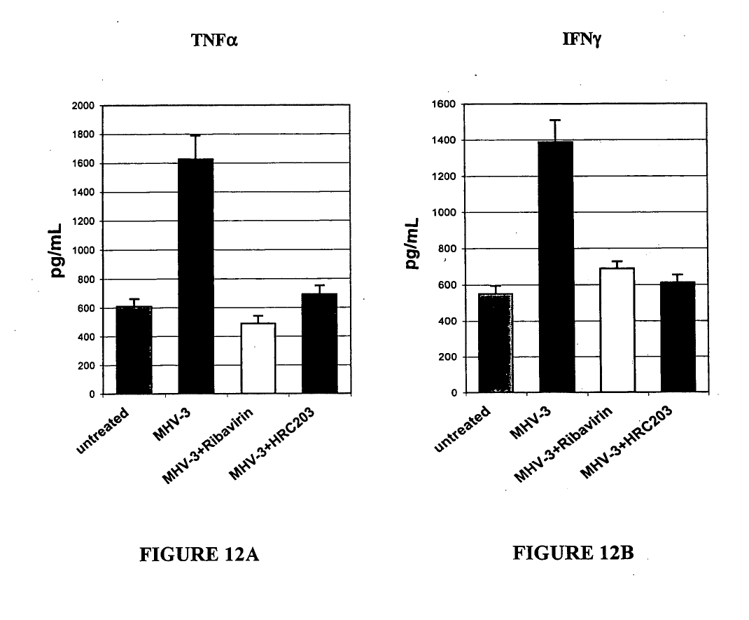

Production of pro-inflammatory mediators including tumor necrosis factor (TNFα) and interferon (IFNγ) were markedly reduced by Hp-Hb-RV as compared with the untreated control. Results are illustrated in FIG. 12 (a) TNFα and (b) IFNγ.

### Example 4—Conjugation of Ara-AMP and Ara-CMP To Hemoglobin

Although hemoglobin-ribavirin was used in the above-noted examples, other ribaivirin-like nucleoside-analogue antivirals could also be used in targeted drug delivery to macrophages and cell containing CD163 receptor.

Preparation of Ara-AMP-Imidazolide (Ara-AMP-lm) and Ara-CMP-Imidazolide (Ara-CMP-lm)

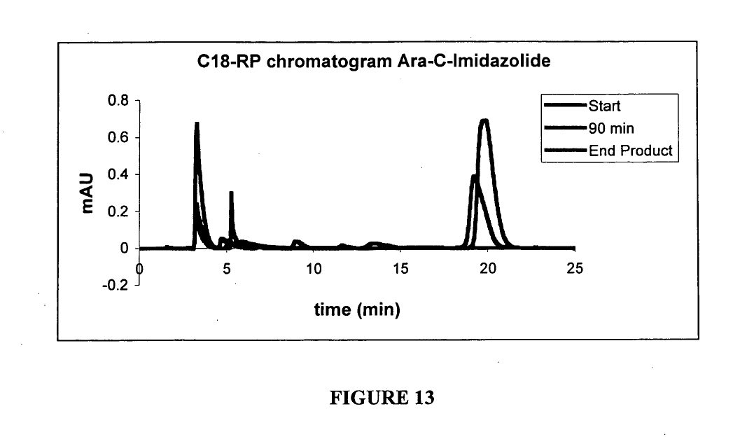

Reactions were conducted under dry N₂ using anhydrous reagents. Solutions of 6.4 mg, ≈20 umol, Ara-AMP in 1 mL DMF or 24.1 mg, ≈90 umol, Ara-CMP in 3 mL DMF were added to 1 ml dry DMF under N₂. Carbonyldiimidazole (CDI), 156 mg, ≈950 umol was dissolved in 4 ml dry DMF. 28.5 mg, 420 mmol, imidazole was dissolved in 2 ml dry DMF. 0.3 ml of the CDI and 0.5 ml of the imidazole solution were added to the Ara-AMP solution. 1 ml of the CDI and 1.5 ml of the imidazole solution were added to the Ara-CMP solution. Reactions were stirred for 3 hours. The formation of the imidazolide was followed by HPLC (C18 RP Aqua column, mobile phase 66 mMol phosphate buffer, pH 7.35, flow 1 ml min, UV abs. at 254 and 280 nm). The peak corresponding to starting nucleotide was converted to a later eluting species (FIG. 13). DMF was evaporated and the crude reaction products and the resulting oils were dissolved in EtOH. Any undissolved material was removed by centrifugation. The EtOH solutions were precipitated with dry ether at −20° C. Precipitates were isolated by centrifugation, washed with ether and dried under N₂, and shown to be pure by HPLC.

Hb-Ara-A and Hb-Ara-C conjugates

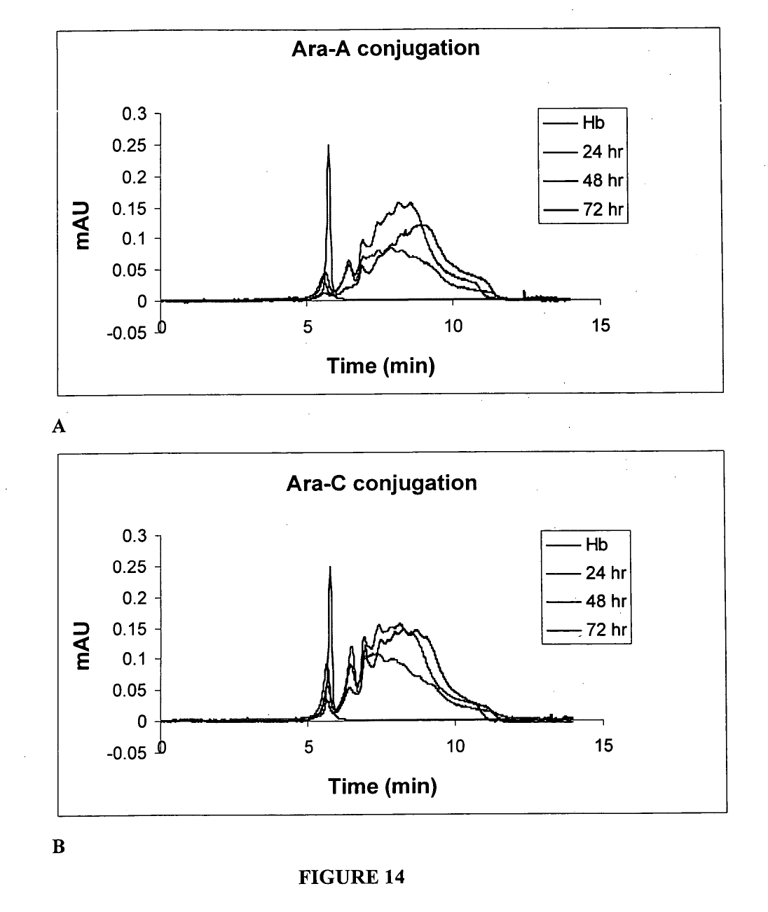

Ara-AMP-lm, 20 umol, was dissolved in 300 ul carbonate buffer, pH 9.3. 125 ul CO-Hb solution was added (10 g/dL, 200 nmol). Ara-CMP-lm, 90 umol, was dissolved in 600 ul carbonate buffer, pH 9.3. 200 ul Hb solution was added (10 g/dL, 320 nmol). The pH of the reaction mixtures was adjusted to pH of approximately 9 , and reaction proceeded at 37° C. Anion exchange chromatography showed the formation of Hb species containing greater negative charge over time, indicating attachment of nucleotide to the Hb (FIG. 14). Reactions were dialyzed against lactated Ringer's solution at 4° C. Pre- and post-dialysis anion exchange profiles were similar, indicating stability of the conjugate during dialysis. Peaks corresponding to non-conjugated nucleotide species were eliminated by dialysis.

Hb concentrations of the conjugates were determined with the Drabkin method and the amount of modification was estimated by measurement of total inorganic phosphate as done for Hb-Ribavirin conjugates. Molar drug ratios (nucleotide:Hb) were 15 and 9 for the Ara-A and Ara-C conjugates, respectively. MALDI-TOF mass spectrometry confirmed the presence of at least 3 nucleotides on each of the alpha and beta chains of the Hb in both conjugates.

TABLE 3

| | Hb concentration | Pi | Molar drug ratio |
|---|---|---|---|
| Conjugate | (g/dL) | (nmol/20 ul) | (nucleotide:Hb) |
| Hb-Ara-A | 1.5 | 69.7 | 15:1 |
| Hb-Ara-C | 1.4 | 35.8 | 9:1 |

### Example 5—Preparation of Haptoglobin Complexes Of Hb-Drug Conjugates

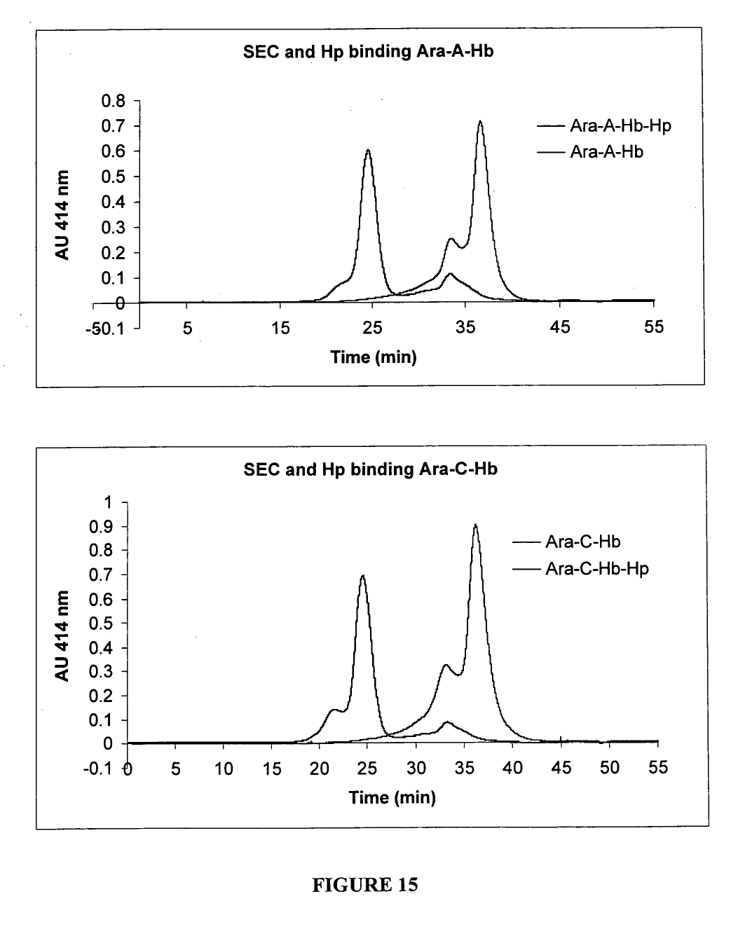

Hb-drug conjugates, prepared according to the preceding example, were combined with at least one equivalent of haptoglobin. Size exclusion chromatography was used to confirm the ability of conjugates to bind haptoglobin. In all cases, conjugates bound, to haptoglobin to form higher molecular weight complexes that eluted earlier than the non-complexed Hb-drug conjugates (FIG. 15).

### Example 6—Specific Uptake Of Labeled Hp-b-AraA Conjugate By CD163—Bearing Cells

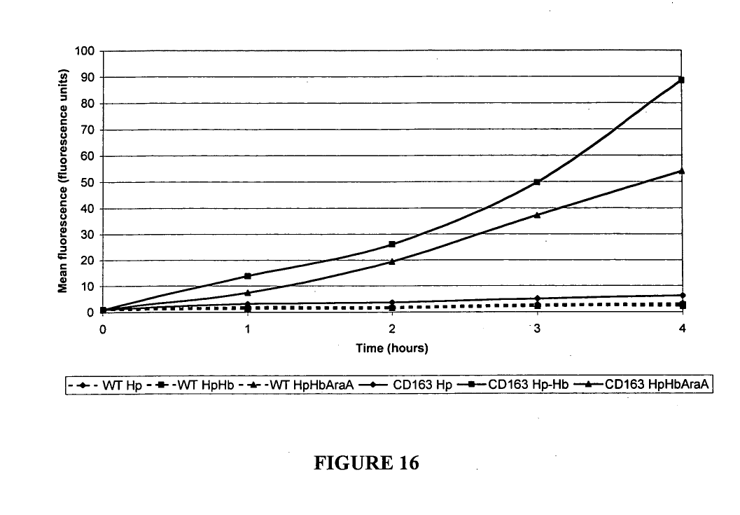

A phosphoramidate-linked conjugate of human hemoglobin (Hb) and Ara-AMP was prepared in a manner similar to that described for preparation of hemoglobin-ribavirin conjugates from Hb and ribavirin-phosphate. Ara-AMP is the 5′-monophosphate form of adenosine arabinoside. Multiple copies of the Ara-AMP were attached to the hemoglobin by this method, and the conjugate is referred to here as Hb-AraA Hb-AraA was complexed to fluorescently-labeled haptoglobin (Fl-Hp). The Fl-Hp was prepared from human Hp (mixed type) using Molecular Probes Alexafluor 488 reagent according to instructions provided by the reagent manufacturer. CD163-expressing CHO cells (CHO-CD163) and wild type CHO cells (CHO-WT, which lack the CD163 receptor) were incubated with the complex at 37° C. in AIM-V for 1 to 4 hours at a Hb concentration of 25 μg/ml. Samples were removed at 1, 2, 3 and 4 hours and assayed by flow cytometry for fluorescence attributable to the Alexafluor label. Time zero corresponds to untreated cells. CHO-WT cells did not take up Fl-Hp, Fl-Hp-Hb or Fl-Hp-Hb-AraA conjugates, while CHO-CD163 cells did take up Fl-Hp-Hb and Fl-Hp-Hb-AraA conjugates over the 4 hour period as indicated by the increasing mean fluorescence of the cell population from 1 to 4 hours (FIG. 16). Therefore, a Hb conjugate of the antiviral drug adenine arabinoside was shown to be selectively targeted to cells bearing CD163, a known Hb-Hp receptor. Also, the attachment of the drug did not prevent the receptor recognition of the Hp-Hb-antiviral complex.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
(HRC 203 = Hp-Hb-Ribavirin)

| | HRC 203 treated mice do not develop anemia | | | | |
|---|---|---|---|---|---|
| Group | Day 1 | Day 2 | Day 3 | Day 4 | Day 5 |
| Hematocrit | | | | | |
| Normal | 41% ± 3% | | | | |
| Control | 41% | N/A | 43% ± 2% | N/A | no animals |
| Ribavirin | 35% | 33% | 33% | N/A | 42% |
| HRC-203 | 38% | 39% | 40% | 40% | 39% |
| Hemoglobin | | | | | |
| Normal | 139 ± 12 | | | | |
| Control | 139 | N/A | 147 ± 7 | N/A | no animals |
| Ribavirin | 119 | 112 | 112 | N/A | 143 |
| HRC-203 | 129 | 133 | 136 | 136 | 133 |

1 A pharmaceutical composition comprising an anti-viral nucleoside analogue and a pharmaceutically acceptable carrier, wherein said carrier is hemoglobin.

2. The pharmaceutical composition of claim 1 wherein the hemoglobin is conjugated to the anti-viral nucleoside analogue to form a hemoglobin-anti-viral nucleoside conjugate.

3. The pharmaceutical composition of claim 2 wherein the conjugate is a hemoglobin-phosphoramidate-anti-viral nucleoside analogue conjugate.

4. The pharmaceutical composition of claim 2 wherein the conjugate is labeled.

5. The pharmaceutical composition of claim 4 wherein the conjugate is fluorescently- or radio-labeled.

6. The pharmaceutical composition of claim 2, wherein the conjugate is capable of binding haptoglobin.

7. The pharmaceutical composition of claim 6, wherein the conjugate is further bound to haptoglobin to form a haptoglobin-hemoglobin-anti-viral nucleoside analogue complex.

8. The pharmaceutical composition of claim 2 wherein the anti-viral nucleoside analogue is selected from the group consisting of AraA, AraC, and ribavirin.

9. The pharmaceutical composition of claim 8, wherein the anti-viral nucleoside analogue is ribavirin.

10. The pharmaceutical composition of claim 1 wherein the hemoglobin directs delivery of the anti-viral nucleoside compound to a CD163 bearing cell or a cell comprising a receptor for hemoglobin or its derivatives.

11. The pharmaceutical composition of claim 10 wherein the cell is a macrophage.

12. The pharmaceutical composition of claim 1, wherein the hemoglobin is non-intramolecularly cross-linked.

13. The pharmaceutical composition of claim 1 wherein the hemoglobin is human hemoglobin.

14. A method of treating a viral infection comprising administering to a subject in need thereof, a pharmaceutical composition of claim 1.

15. The method of claim 14, wherein the anti-viral nucleoside analogue is ribavirin.

16. The method of claim 15, wherein the viral infection is selected from the group consisting of Hepatitis C, HIV, SARS, coronavirus, and RSV.

17. The method of claim 14, wherein the pharmaceutical composition is administered intravenously.

18. A method of treating a viral or non-viral condition that is modulated through macrophages, comprising administering to a patient in need thereof, a pharmaceutical composition of claim 1.

19. A method of reducing the incidence of hemolytic anemia associated with anti-viral nucleoside therapy comprising administration of an effective amount the pharmaceutical composition of claim 2.

20. The method of treating a viral infection in accordance with claim 14 comprising administering the pharmaceutical composition in combination with IFN or PEG-IFN

21. The method of claim 20, wherein the viral infection is hepatitis C.

22. A method of treating a non-viral infection comprising administering to patient in need thereof, a pharmaceutical composition of claim 1.
